(19) United States
(12) Patent Application Publication
(10) Pub. No.: US 2003/0109447 A1
(43) Pub. Date: Jun. 12, 2003

(54) Methods of use of recombinant vasoactive protein from salivary gland of the black fly

(75) Inventors: Mary Smith Cupp, Auburn, AL (US); Eddie Wayne Cupp, Auburn, AL (US); Steven F. Swaim, Auburn, AL (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Auburn University, Auburn, AL (US)

(21) Appl. No.: 10/288,740

(22) Filed: Nov. 6, 2002

Related U.S. Application Data
(63) Continuation-in-part of application No. 10/218,699, filed on Aug. 14, 2002.
Application No. 10/218,699 is a division of application No. 09/702,647, filed on Oct. 31, 2000, now Pat. No. 6,500,420.
Application No. 09/702,647 is a division of application No. 09/036,355, filed on Mar. 6, 1998, now Pat. No. 6,162,785.
(60) Provisional application No. 60/040,418, filed on Mar. 13, 1997.

(51) Int. Cl. A61K 38/17; C12P 21/02

(52) U.S. Cl. 514/12; 435/69.1

(57) ABSTRACT

The invention is drawn to vasodilative proteins from the salivary glands of the species of Simulium. The protein additionally has immunomodulating activities. Methods for recombinant production of the protein as well as biomedical uses are provided.

### CROSS-REFERENCE TO RELATED APPLICATIONS

This application is a continuation-in-part of copending U.S. patent application Ser. No. 10/218,699, filed Aug. 14, 2002, which is a divisional application of U.S. patent application Ser. No. 09/702,647, filed Oct. 31, 2000, which is a divisional application of U.S. patent application Ser. No. 09/036,355 filed Mar. 6, 1998, which claims the benefit of U.S. Provisional Application No. 60/040,418, filed Mar. 13, 1997.

### FIELD OF THE INVENTION

The invention relates to the production of recombinant proteins and their use in biomedical therapies.

### BACKGROUND OF THE INVENTION

Hypertension is the most common cardiovascular disease. Many people in the United States suffer from what is commonly referred to as “high blood pressure,” i.e., a systolic and/or diastolic blood pressure above 140/90.

Elevated arterial pressure causes pathological changes in the vasculatury and hypertrophy of the left ventricle. As a consequence, hypertension has many deleterious effects on the body. For example, it is the principal cause of stroke, leads to disease of the coronary arteries with myocardial infarction and sudden cardiac death, and is a major contributor to cardiac failure, renal insufficiency, and dissecting aneurism of the aorta.

Pharmacological treatment of patients with high blood pressure will reduce morbidity, disability, and mortality from cardiovascular disease. Effective antihypertensive therapy will almost completely prevent hemorrhagic strokes, cardiac failure, and renal insufficiency due to hypertension. Overall, there is a marked reduction in total strokes.

Antihypertensive drugs can be classified according to their sites or mechanisms of action. Arterial pressure is the product of cardiac output and peripheral vascular resistance. Thus, such pressure can be lowered by actions of drugs on either the peripheral resistance or the cardiac output, or both. Drugs may reduce the cardiac output by either inhibiting myocardial contractility or decreasing ventricular filling pressure.

Reduction in ventricular filling pressure may be achieved by actions on the venous tone or on blood volume via renal effects. Drugs can reduce peripheral resistance by acting on smooth muscle to cause relaxation of resistance vessels or by interfering with the activity of systems that produce constriction of resistance vessels.

Vasodilators are a class of drugs which are commonly employed in the therapy of heart failure, high blood pressure, and other various conditions characterized by constricted blood vessels. Such conditions include Raynaud's syndrome, certain post-surgical complications of brain surgery involving subarachnoid hemorrhage, heart failure, angina pectoris, and hypertension.

Proteins from biting insects, particularly blood-feeding arthropods, have been shown to contain numerous pharmacologically-active substances, including vasodilating substances. The saliva from such insects contains such substances to counteract many of the host's hemostatic defenses. Among these secretions are the potent vasodilating substances that heighten blood flow to the feeding site.

The salivary components responsible for vasodilation are extremely varied as revealed by the recent characterization of purified factors from several genera. Of several species of ticks analyzed, the saliva of each contained a lipid-derived prostaglandin that could account for vasodilative activity. Vasodilators play a role in skin-associated immune response.

Specific immunity has evolved as a sophisticated defense mechanism of higher organisms. In humans, cell-mediated immunity and humoral immunity are the two major mechanisms. Both of these responses have a high level of specificity directed to antigenic epitopes expressed on molecular components of foreign agents.

There are several clinical settings where it is desirable to suppress an immune response. These situations include organ transplantation, treatment of autoimmune disorders, and prevention of Rh hemolytic disease of the newborn. Because of the importance of treating such diseases and disorders, agents which are capable of modulating the immune response are desirable.

There are several clinical settings where it is desirable to enhance blood flow, such as in wound healing and in the treatment of diseases which affect blood flow. For example, decreased blood flow in diabetic patients can result in nonhealing, necrotic diabetic foot ulcers. Such ulcers are estimated to contribute to approximately 50,000 lower extremity amputations per year in the United States (Reiber et al. (1995) “Lower extremity foot ulcers and amputations in diabetes,” in Diabetes in America, ed. National Diabetes Data Group (National Institutes of Health)). The health care costs associated with these problems have been estimated to be in excess of $1 billion per year (Pham et al. (1999) Wounds 11: 79). Decubital (pressure) ulcers are estimated to affect 1 million people each year, leading to an annual cost of approximately $1.6 billion. Other wounds are also associated with low blood flow such as, for example, diabetic retinopathy. The intractable nature and attendant costs of treating such chronic nonhealing wounds requires the development of new treatment modalities that address the underlying physiological basis for the pathology.

Thus, because of the importance of providing therapies for hypertension and problems associated with low blood flow, potent vasodilators are needed. Additionally, agents which are capable of aiding in wound healing and preventing ulcer formation are desirable.

### SUMMARY OF THE INVENTION

Purified vasoactive proteins from the salivary glands of the blood-feeding black fly, Simulium spp., are provided. The proteins find use in biomedical therapies, particularly where peripheral resistance and stenoses are problems. The proteins are also useful as regulators of the immune response and as promoters of wound healing.

The nucleotide sequence encoding the proteins, as well as methods for producing recombinant protein, are additionally provided.

### BRIEF DESCRIPTION OF THE DRAWINGS

FIG. 1 shows the presence of erythema in NZW rabbits following intradermal injection of SGE of female S. vittatum or rSVEP.

FIG. 2 shows the dose-related perfusion response to SVEP of NZW rabbits. (See Example 2). In this experiment, varying concentrations of SVEP were injected intradermally into intact skin in order to quantitate blood flow response using Laser Doppler Perfusion Imaging (LDPI) measurements. Increase in perfusion is shown as percentage of the Phosphate Buffered Saline (PBS)-treated control as a function of time (in hours). Three dosages of SVEP were used: 0.3 micrograms (horizontally-striped bars); 1.5 micrograms (open bars); and 3.0 micrograms (diagonally-striped bars).

FIG. 3 shows the perfusion response to SVEP in beagle dog skin. Percentage increase over perfusion at time 0 is shown for five dogs with both PBS (control) and SVEP-treated wounds. (See Example 2).

FIG. 4 shows the secretion of nitric oxide by macrophage-like cells (cell line RAW 264.7) in response to increasing concentrations of IFN-γ. (See Example 3). Cells were treated with 0, 50, 100, 200, 300, or 500 Units/ml IFN-γ. Open bars represent data collected at 24 hours, and horizontally-striped bars represent data collected at 48 hours. Nitric oxide secretion was monitored by measuring nitrite, which is released by activated cells as the end-product of nitric oxide. All concentrations of IFN-γ tested caused macrophage activation (i.e., the initial inflammatory response), but activation was maximal at an IFN-γ concentration of 100 Units/ml.

The FIG. 5 data demonstrate that SVEP can modulate the response of macrophage-like cells to IFN-γ. In this experiment, macrophage-like RAW 264.7 cells were treated for two hours with increasing concentrations of SVEP of 0, 0.25, 0.50 or 1 microgram per ml (i.e., 0, 16, 33, or 65 nanomolar), followed by the addition of 100 Units/ml of IFN-γ. (See Example 3). Inflammatory macrophage response was assessed by measuring the nitric oxide (NO) concentration of cultures after 48 hours. The FIG. 5 graph shows percent increase over the control treatment (i.e., treatment with 0 micrograms per ml SVEP) as a function of the SVEP concentration used (in micrograms per ml). The data show that SVEP has an inhibitory effect on NO production which is maximal at 1 microgram/ml SVEP and begins to reverse at higher concentrations, thereby indicating that SVEP modulates the response of macrophage-like cells to IFN-γ.

FIG. 6 shows that SVEP increases blood flow in skin of beagle dogs. Percent increase in blood flow is shown as a function of the dose of SVEP at various times following treatment. Stippled bars represent data gathered at 0.5 hours; horizontally-striped bars represent data gathered at 24 hours; open bars represent data gathered at 48 hours; and diagonally-striped bars represent data gathered at 72 hours (see Example 6).

FIG. 7 shows the results of breaking strength tests (see Example 7). Percent increase in breaking strength over PBS control is shown for wounds on each of five dogs.

The breaking strength of closed wounds was measured at day 5. SVEP treatment improved the breaking strength of closed wounds (Wilcoxon Signed Rank Test, n=5, p=0.043). The median increase in breaking strength due to SVEP treatment was 48%.

FIG. 8 shows results for total healing of open wounds as described in Example 7. Percentage of total wound healing is shown as a function of days of healing. Lines marked with open circles and open triangles represent results from wounds treated with subcutaneous injection on days 0, 3, 6, 9, and 12. The line marked with open circles shows data from treatment with PBS (control) and the line marked with open triangles shows data from SVEP treatment with 5 micrograms of SVEP. Lines marked with open diamonds and open squares represent results from wounds treated with intradermal injection on day 0 and then with subcutaneous injection on days 3, 6, 9, and 12. The line marked with open diamonds shows data from SVEP treatment, and the line marked with open squares shows data from the PBS control treatment.

FIG. 9 shows the effect of SVEP on cell proliferation of RAW 264.7 cells in vitro as measured with an “MTT” assay (see Example 4). The graph shows the absorbance values of cells as a function of increasing SVEP concentration in nanomolar amounts. Closed circles are data from cell cultures in the absence of IFN-γ, and open circles are data from cell cultures treated with IFN-γ.

FIG. 10: An assay was performed on the clarified, cell-conditioned medium from cells treated with IFN-γ and SVEP to quantitate nitrite, the metabolite of NO, in the media (see Example 4). Nitric oxide concentration (nanomolar) is shown as a function of SVEP concentration (nanomolar), and the results indicate that NO production was inhibited by SVEP at a particular concentration.

FIG. 11: ELISA analysis was performed to quantitate the IGF-1 response of cells treated with IFN-γ and SVEP (see Example 4). The IGF-1 concentration (picomolar) is shown as a function of SVEP concentration (nanomolar).

### DETAILED DESCRIPTION OF THE INVENTION

Methods and compositions for use as therapeutic vasodilating agents, i.e., as regulators of blood pressure are provided. The agents can be used to regulate blood flow to a wound site promoting wound healing. Additionally, the compositions of the invention can be utilized to modulate the immune response.

The compositions of the invention comprise vasoactive proteins from the salivary glands of the blood-feeding black fly. The proteins exhibit vasodilative activity and wound healing promoting properties, as well as the capacity to suppress certain immune responses in a mammal. Substantially purified preparations of the proteins are provided.

Such substantially purified preparations include protein substantially free of any compound normally associated with the protein in its natural state. Such proteins can be assessed for purity by SDS-PAGE, chromatography, electrophoresis or other methods known in the art. See, M. P. Deutscher (ed.), Guide to Protein Purification, Academic Press, Inc. (1990).

The terms substantially pure or substantially purified are not meant to exclude artificial or synthetic mixtures of the protein with other compounds. It is recognized that the vasoactive proteins of the present invention include those proteins homologous to, and having essentially the same biological properties as, the vasoactive protein described herein, and particularly the protein disclosed herein in SEQ ID NO: 2. This definition is intended to encompass natural allelic variations in the genes.

The invention additionally encompasses the nucleotide sequences which encode the proteins of the invention. The nucleotide sequence of the coding sequence from S. vittatum is provided in SEQ ID NO: 1. Additionally, cloned genes of the present invention can be of other species of origin. Thus, DNAs which hybridize to the nucleotide sequence of the vasoactive gene from the black fly are also an aspect of this invention. Conditions which will permit other DNAs to hybridize to the DNA disclosed herein can be determined in accordance with known techniques. For example, hybridization of such sequences may be carried out under conditions of reduced stringency, medium stringency or even stringent conditions (e.g., conditions represented by a wash stringency of 35-40% formamide with 5× Denhardt's solution, 0.5% SDS and 1×SSPE at 37° C.; conditions represented by a wash stringency of 40-45% formamide with 5× Denhardt's solution, 0.5% SDS, and 1×SSPE at 42° C.; and conditions represented by a wash stringency of 50% formamide with 5× Denhardt's solution, 0.5% SS and 1×SSPE at 42° C., respectively, to DNA encoding the vasoactive genes disclosed herein in a standard hybridization assay. See J. Sambrook et al., Molecular Cloning, A Laboratory Manual (2d Ed. 1989) (Cold Spring Harbor Laboratory).

In general, sequences which code for the vasoactive protein and hybridize to the nucleotide sequence disclosed herein will be at least 75% homologous, 85% homologous, and even 95% homologous or more with the sequences. Thus, sequences of the invention will share at least 75% sequence identity or 80%, 85%, 90%, 93%, 95%, 96%, 97%, 98%, or 99% or higher sequence identity with the exemplary disclosed sequences. Sequence identity for DNA sequences can be calculated manually by comparison to the full-length exemplary disclosed sequences or may be calculated manually in comparison to a corresponding subportion of the exemplary disclosed sequences where fragments are being compared. In addition, computer programs which use alignment algorithms are known in the art and may be used to calculate sequence identity, such as, for example, BLAST in the Wisconsin Genetics Software Package, Version 8 (available from Accelrys, Inc., 9685 Scranton Road, San Diego, Calif. 92121-3752, USA), using default parameters.

Further, the amino acid sequences of the vasoactive proteins isolated by hybridization to the DNA's disclosed herein are also an aspect of this invention. The degeneracy of the genetic code, which allows different nucleic acid sequences to code for the same protein or peptide, is well known in the literature. See, e.g., U.S. Pat. No. 4,757,006.

The hybridization probes may be cDNA fragments or oligonucleotides, and may be labeled with a detectable group as known in the art. Pairs of probes which will serve as PCR primers for the vasoactive gene or a protein thereof may be used in accordance with the process described in U.S. Pat. Nos. 4,683,202 and 4,683,195.

It is recognized that the nucleotide and peptide sequences of the invention may be altered in various ways including amino acid substitutions, deletions, truncations, and insertions. Methods for such manipulations are generally known in the art. For example, amino acid sequence variants of the peptides and proteins can be prepared by mutations in the DNA. Methods for mutagenesis and nucleotide sequence alterations are well known in the art. See, for example, Kunkel, T. (1985) Proc. Natl. Acad. Sci. USA 82:488-492; Kunkel et al. (1987) Methods in Enzymol. 154:367-382; U.S. Pat. No. 4,873,192; Walker and Gaastra (eds.) Techniques in Molecular Biology, MacMillan Publishing Company, NY (1983) and the references cited therein. Thus, the nucleotide sequences of the invention include both the naturally occurring sequences as well as mutant forms. Likewise, the peptides and proteins of the invention encompass both naturally occurring and modified forms thereof. Such variants will continue to possess the desired activity. Obviously, the mutations that will be made in the DNA encoding the variant must not place the sequence out of reading frame and preferably will not create sequences deleterious to expression of the gene product. See, EP Patent Application Publication No. 75,444.

Thus proteins of the invention include the naturally occurring forms as well as variants thereof. These variants will be substantially homologous and functionally equivalent to the native protein. A variant of a native protein is “substantially homologous” to the native protein when at least about 80%, more preferably at least about 90%, and most preferably at least about 95% of its amino acid sequence is identical to the amino acid sequence of the native protein. Thus, a variant of a native protein is “substantially homologous” to the native protein when at least about 80%, 85%, 90%, or 95% or 96%, 97%, 98%, 99%, or more of its amino acid sequence is identical to the amino acid sequence of the native protein. A variant may differ by as few as 1, 2, 3, or 4 amino acids. Sequence identity may be calculated manually by inspection with regard to the complete exemplary sequence or a subportion thereof. In addition, sequence identity may be calculated using a computer program implementation of a mathematical alignment algorithm, such as BLAST in the Wisconsin Genetics Software Package, Version 8, using default parameters.

By “functionally equivalent” is intended that the sequence of the variant defines a chain that produces a protein having substantially the same biological activity as the native protein of interest. Such functionally equivalent variants that comprise substantial sequence variations are also encompassed by the invention. Thus a functionally equivalent variant of the native protein will have a sufficient biological activity to be therapeutically useful. By “therapeutically useful” is intended effective in achieving a therapeutic goal as discussed in more detail below. Fragments of the exemplary disclosed sequences are also provided. By “fragment” is intended a portion of a nucleotide or amino acid sequence. Proteins which are fragments of an amino acid sequence retain at least one activity of a protein having the native amino acid sequence, and fragments of a nucleotide sequence encode proteins that retain at least one activity of a protein having the native amino acid sequence. A fragment of an SVEP nucleotide sequence that encodes a biologically active SVEP protein of the invention will encode at least 10, 20, 30, 40, 50, 60, 70, 80, 90, or 100 contiguous amino acids of SEQ ID NO:2, or the complete 105 contiguous amino acids of SEQ ID NO:2.

Methods are available in the art for determining functional equivalence. Biological activity can be measured using assays specifically designed for measuring activity of the native protein, including assays described in the present invention. Additionally, antibodies raised against the biologically active native protein can be tested for their ability to bind to the functionally equivalent variant, where effective binding is indicative of a protein having conformation similar to that of the native protein.

DNA sequences can also be synthesized chemically or modified by site-directed mutagenesis to reflect the codon preference of the host cell and increase the expression efficiency.

The proteins of the invention can be “engineered” in accordance with the present invention by chemical methods or molecular biology techniques. Molecular biology methods are most convenient since proteins can be engineered by manipulating the DNA sequences encoding them. Genomic DNA, cDNA, synthetic DNA, and any combination thereof may be used for this purpose. Genomic DNA sequences or cDNA sequences encoding proteins can be isolated based on the amino acid sequence of proteins or certain protein properties. Many methods of sequence isolation are known in the art of molecular biology. See particularly Sambrook et al. (1989) Molecular Cloning: A Laboratory Manual (Cold Spring Harbor Laboratory Press, Plainview, N.Y.), herein incorporated by reference.

Once the nucleotide sequences encoding the vasoactive proteins of the invention have been isolated, they can be manipulated and used to express the protein in a variety of hosts including other organisms, including microorganisms.

Once the nucleotide sequence is identified and known, those skilled in the art can produce large quantities of the protein for therapeutic use. Accordingly, recombinant protein and methods for producing the recombinant protein are encompassed by the present invention. In this manner, the nucleotide sequence encoding the vasoactive protein can be utilized in vectors for expression in various types of host cells, including both procaryotes and eucaryotes, to produce large quantities of the protein, or active analogues, or fragments thereof, and other constructs capable of inducing vasodilation or temporarily suppress the immune response in a mammal.

Generally, methods for the expression of recombinant DNA are known in the art. See, for example, Sambrook et al. Molecular Cloning, Cold Spring Harbor Laboratory (1989). Additionally, host cells and expression vectors, such as the baculovirus expression vector may be employed in carrying out the present invention, as described in U.S. Pat. Nos. 4,745,051 and 4,879,236. In general, a baculovirus expression vector comprises a baculovirus genome containing the gene to be expressed inserted into the polyhedron gene at a position ranging from the polyhedron transcriptional start signal to the ATG start site and under the transcriptional control of a baculovirus polyhedron promoter.

A broad variety of suitable procaryotic and microbial vectors are available.

Likewise, the promoters and other regulatory agents used in expression of foreign proteins are available in the art. Promoters commonly used in recombinant microbial expression vectors are known in the art and include the beta-lactamase (penicillinase) and lactose promoter systems (Chang et al. (1978) Nature 275: 615 and Goeddel et al. (1979) Nature 281: 544); a tryptophan (TRP) promoter system (Goeddel et al. (1980) Nucleic Acids Res. 8: 4057 and the EPO Application Publication No. 36,776); and the Tac promoter (DeBoer et al. (1983) Proc. Natl. Acad. Sci. USA 80: 21). While these are commonly used, other microbial promoters are available. Details concerning nucleotide sequences of many have been published, enabling a skilled worker to operably ligate them to DNA encoding the protein in plasmid or viral vectors. See, for example, Siedenlist et al. (1980) Cell 20:269.

Eukaryotic microbes such as yeast may be transformed with suitable protein-encoding vectors. See, e.g., U.S. Pat. No. 4,745,057. Saccharomyces cerevisiae is the most commonly used among lower eukaryotic host microorganisms, although a number of other strains are commonly available. Yeast vectors may contain an origin of replication from the 2 micron yeast plasmid or an autonomously replicating sequence (ARS), a promoter, DNA encoding the desired protein, sequences for polyadenylation and transcription termination, and a selection gene. An exemplary plasmid is YRp7 (Stinchcomb et al. (1979) Nature, 282:9; Kingsman et al. (1979) Gene, 7:141; Tschemper et al. (1980) Gene, 10:157). This plasmid contains the trp1 gene, which provides a selection marker for a mutant strain of yeast lacking the ability to grow in tryptophan, for example ATCC No. 44076 or PEP4-1 (Jones (1977) Genetics 85: 12). The presence of the trp1 lesion in the yeast host cell genome then provides an effective environment for detecting transformation by growth in the absence of tryptophan.

Suitable promoting sequences in yeast vectors include the promoters for metallothionein, alcohol dehydrogenase, adenylate cyclase, 3-phospho-glycerate kinase (Hitzeman et al. (1980) J. Biol. Chem. 255: 2073) and other glycolytic enzymes (Hess et al. (1968) J. Adv. Enzyme Reg. 7: 149; and Holland et al. (1978) Biochemistry 17: 4900) such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Suitable vectors and promoters for use in yeast expression are further described in R. Hitzeman et al. EPO Publication. No. 73,657.

The compositions of the present invention can be formulated into pharmaceutical preparations for therapeutic use. As a vasodilator, the compositions find use for treatment of atherosclerosis of extremities, heart failure, hypertension, peripheral resistance, stenoses, and the like, particularly peripheral vasodilation. Thus, the compositions find use in treating diseases and disorders associated with both the central circulatory system and the peripheral circulatory system.

The compositions of the invention can also be used to suppress temporarily the immune system. In this manner, a mammal can be desensitized to the effects of an immunogen by parenteral administration of the vasoactive protein, active analogs or fragments thereof. For modulating the immune system, the proteins can be utilized to inhibit or prevent the development of antibodies or cellular immunity to a protein, to treat graft rejection, inflammation, autoimmune diseases, and the like.

The compositions of the invention find use as promoters of wound healing. Application to the wound site results in an increased rate of healing and/or in improved healing, such as for example, healing with reduced, scar tissue. One goal of wound management is to attain rapid healing of open wounds by second intention (i.e., the union by closure of a wound with granulations which form from the base and both sides toward the surface of the wound). A major determinant in rate of healing and strength of the wound is blood supply to the healing tissues. While the invention is not limited by a particular mechanism of operation, the blood supply provides the primary means by which the cells, their associated growth factors, and oxygen are delivered to the healing tissues. In addition, unobstructed blood flow is important for nutrient delivery, the delivery of regulatory hormones, and the removal of metabolic waste from tissues. Because impaired blood flow is both an outcome and a cause of injury and disease, wound healing can be promoted by enhancing blood supply to the wounded area.

Enhanced blood supply may result from increased blood flow through existing vessels (e.g., vasodilation) or by an increase in new vessels (angiogenesis). Thus, administration of SVEP, for example, by, intralesion injection, is thought to provide a simple means of increasing oxygen to wound tissues and promoting or enhancing wound healing via vasodilation. From this description, it will be appreciated that by “vasoactive” is intended that a compound exerts an effect on the caliber of blood vessels, and vasoactive activity may be measured by observing any phenomena associated with change in caliber of blood vessels, such as, for example, perfusion of blood into a tissue or enhanced wound healing.

While the invention is not bound by any particular mechanism of action, another way in which SVEP may enhance or promote wound healing is by affecting macrophage cellular response to IFN-γ. In a wound, control of macrophage phenotype appears to be a pivotal event in the sequential steps required to move from the appropriate initial inflammation stage to the later tissue repair stage. The initial inflammation stage is associated with the induction of iNOS (inducible nitric oxide synthase) and secretion of NO (nitric oxide), while the later tissue repair stage is associated with the inactivation of iNOS and secretion of other repair-associated molecules, including IGF-1. That is, nitric oxide production is a marker for the inflammatory macrophage response phenotype, while IGF-1 production is a marker for the non-inflammatory or “healing” macrophage response phenotype and is associated with healing wounds. SVEP action decreases NO production that is associated with an “inflammatory” phenotype macrophage while concomitantly increasing IGF-1 secretion, reflecting a switch to a “healing” macrophage phenotype (see Examples 3 and 4). This effect of SVEP shows an interaction with IFN-γ (see Example 3). It is understood that reduction in inflammation and change in macrophage cellular response are aspects of the immune response, which is thereby shown to be modulated by SVEP treatment.

The most sensitive response of surgically-created wounds to treatment with SVEP was a decrease in tissue necrosis, and therefore SVEP may promote wound healing in part by modulating macrophage activation responses to decrease the inflammatory response. In this manner, the compounds of the invention enhance or promote wound healing by improving at least one aspect of wound healing, for example, by enhancing macrophage cellular response to promote the “healing” macrophage phenotype and suppress the inflammatory phenotype. Other aspects of wound healing that can be improved by the compounds of the invention include, but are not limited to, increasing cellular proliferation, decreasing inflammation, decreasing tissue necrosis, and/or enhancing vasodilation. These aspects of wound healing may be measured in any suitable manner; for example, whether inflammation is decreased may be assessed by measuring macrophage phenotype or it may also be assessed by preparing tissue sections and scoring for degree of tissue necrosis. One of skill is aware of assays and techniques for measuring aspects of wound healing.

The compositions of the invention can be used alone or in combination with other vasoactive and/or therapeutic agents. A therapeutic agent is any agent that is useful for treating a wound. Thus, increased blood supply to a wound could be provided by vasodilation as well as by angiogenesis in the wound vicinity, and the vasoactive compositions of the invention could be used in combination with, for example, angiogenic compositions to provide an improvement in blood supply to the wound. This increased blood supply to the wound provided by the vasoactive compounds of the invention may result in increased delivery of systemic compounds to the wound, for example, antibiotics.

Thus, SVEP may act to enhance delivery of other compounds to wounds, and the vasoactive compounds of the invention could be used in combination with other agents, for example, antibiotics to enhance the treatment of the wound. (See, for example, Cross et al. (1996) Antimicrob. Agents Chemother. 40: 1703). Other agents may include, for example, antibiotics, or may also include factors such as, for example: insulin-like growth factor-1 (IGF-1); platelet-derived growth factor (PDGF); transforming growth factor beta (TGF-β); transforming growth factor alpha (TGF-α); tumor necrosis factor-alpha (TNF α); interleukin-1 (IL-1); basic fibroblast growth factor (b-FGF); and interferon-gamma (IFN γ). Other agents are known in the art.

Increased tissue perfusion resulting from vasodilation associated with the compounds of the invention provides a unique delivery of these factors to wounds and helps assure perfusion of severely traumatized wound tissues. In this manner, treatment with the compositions of the invention finds use in stimulating the healing of chronic wounds associated with extensive cellular necrosis such as, for example, venomous bite wounds, decubital ulcers, ocular wounds, and diabetic foot lesions. In addition, the compositions of the invention find use in any application in which increased vasodilation or blood perfusion may be beneficial, for example, wounds due to trauma, burn, radiation, frost bite, pressure, and surgery. Thus, by “wound” is intended tissue damage such that the tissue is in an abnormal condition. For example, a wound may result from an injury, such as blunt force trauma, or it may result from a disease or disorder, such as hypertension or diabetes. Wounds may occur in any tissue type and at any developmental stage, and more than one wound may be present in the patient to be treated. Thus, for example, a wound may be an ocular wound resulting from traumatic eye injury or diabetic retinopathy or both. Ocular wounds also include but are not limited to eyelid wounds, corneoscleral wounds, and anterior uveitis.

The vasoactive compositions can be formulated according to known methods to prepare pharmaceutically useful compositions, such as by admixture with a pharmaceutically acceptable carrier vehicle. Suitable vehicles and their formulation are described, for example, in Remington's Pharmaceutical Sciences 16th ed., Osol, A. (ed.), Mack Easton Pa. (1980). In order to form a pharmaceutically acceptable composition suitable for effective administration, such compositions will contain an effective amount of the vasoactive protein, either alone, or with a suitable amount of carrier vehicle.

Additional pharmaceutical methods may be employed to control the duration of action. Controlled release preparations may be achieved by the use of polymers to complex or absorb the antiviral compositions. The controlled delivery may be exercised by selecting appropriate macromolecules (for example, polyesters, polyamino acids, polyvinyl pyrrolidone, ethylene-vinylacetate, methylcellulose, carbosymethylcellulose, or protamine sulfate). The rate of drug release may also be controlled by altering the concentration of such macromolecules.

Another possible method for controlling the duration of action comprises incorporating the therapeutic agents into particles of a polymeric substance such as polyesters, polyamiono acids, hydrogels, poly(lactic acid) or ethylene vinylacetate copolymers. Alternatively, it is possible to entrap the therapeutic agents in microcapsules prepared, for example, by coacervation techniques or by interfacial polymerization, for example, by the use of hydroxymethyl cellulose or gelatin-microcapsules or poly(methylmethacrylate) microcapsules, respectively, or in a colloid drug delivery system, for example, liposomes, albumin, microspheres, microemulsions, nanoparticles, nanocapsules, or in macroemulsions. Such teachings are disclosed, for example, in Remington's Pharmaceutical Sciences (1980).

It is contemplated that the inhibitory compositions of the present invention will be administered to an individual in therapeutically effective amounts. That is, in an amount sufficient to enhance or promote wound healing, regulate blood pressure and/or suppress the immune response. The effective amount of the composition will vary according to the weight, sex, age, and medical history of the individual. Other factors which influence the effective amount may include, but are not limited to, the severity of the patient's condition, the stability of the vasoactive protein, the kinetics of interaction in the recipient, previous exposure to the vasoactive protein, kidney or other disease, etc. Typically, for a human subject, an effective amount will range from about 0.1 ng to about 100 mg, specifically, from about 1 ng to about 10 mg, more specifically from about 10 ng to about 1 mg. Those of skill in the art, i.e., medical or veterinary practitioners, are aware of routine methods for determining an appropriate rate or dose of administration to achieve the desired result of therapeutic benefit. By “patient” is intended any subject in need of treatment that may benefit from treatment with the compounds of the invention. A patient may be a mammal, such as a human patient, or a dog, horse, cow, sheep, etc. A patient may also be a bird, reptile, amphibian, fish, or other organism.

Thus, by “therapeutically effective” is intended that an aspect of the wound, disease, or disorder, is improved when treated with a compound of the invention compared to a similar wound, disease, or disorder that is not treated with a compound of the invention. By “therapeutically effective amount” is intended the amount of the compound of the invention required to improve at least one aspect of the wound, disease, or disorder. By “enhances wound healing” or “promotes wound healing” is intended that at least one aspect of wound healing is improved when a wound is treated with a compound of the invention in comparison to an untreated wound or a wound treated without a compound of the invention. The aspect of the wound, disease, or disorder may be improved by 5%, 10%, 14%, 15%, 20%, 25%, 30%, 40%, 50%, 60%, 70%, 80%, 90%, or 100% or more when treated with a compound of the invention.

Aspects of the wound that are improved include any measurable phenomenon that is associated with improved condition, i.e., with the healing of a wound. For example, aspects of wounds that are improved with wound healing include increase in tissue perfusion, increase in wound breaking strength, increase in cellular proliferation, decrease in inflammation and/or inflammatory response, and decrease in tissue necrosis. In this manner, the compositions and methods of the invention find further use in controlling or improving these individual aspects or phenomenon that typically, but not always, are associated with wound healing. Thus, the compositions and methods of the invention are useful in modulating or increasing or decreasing the macrophage inflammatory response, inflammation, tissue necrosis, cellular proliferation, etc. in the absence of a wound as well as in enhancing wound healing. By “modulating” is intended that a measurable process or result is increased or decreased. By “increasing” or “decreasing” is intended that the process or result is measurably changed from an appropriate control using any suitable assay. Thus, a process or result may be increased or decreased by 5%, 10%, 14%, 15%, 20%, 25%, 30%, 40%, 50%, 60%, 70%, 80%, 90%, or 100% or more when treated with a compound of the invention. For example, when treated with a compound of the invention, the inflammatory response of macrophage-like cells may be decreased by 5% as evaluated by quantitation of IGF-1 production as described in Example 4.

The pharmaceutically prepared inhibitory compositions of the invention may be provided to a patient by means well-known in the art. Such means of introduction include, for example, oral means, intranasal means, subcutaneous means, intramuscular means, topical administration, injection means, intradermal means, intravenous means, intraarterial means, or parenteral means. Thus, the compositions may be introduced into the lesion by injection. Other compounds or compositions to be used in combination with the compositions of the invention may be administered in any manner, and such administration may precede, follow, and/or occur at the same time as the administration of the compositions of the invention.

The vasoactive proteins of the present invention may be dissolved in any physiologically tolerated liquid in order to prepare an injectable bolus. It is generally preferable to prepare such a bolus by dissolving the molecule in normal saline.

The following examples are offered by way of illustration and not by way of limitation. Many modifications and other embodiments of the invention will come to mind in one skilled in the art to which this invention pertains having the benefit of the teachings presented herein in the descriptions and the associated drawings. Therefore, it is to be understood that the invention is not to be limited to the specific embodiments disclosed. Although specific terms are employed, they are used in a generic and descriptive sense only and not for purposes of limitation, and modifications and embodiments are intended to be included within the scope of the appended claims.

### Experimental

### EXAMPLE 1

### Isolation and Characterization of Simulium vittatum Salivary Gland Erythema Protein (SVEP)

Salivary gland extracts (SGE) of several Simulium were shown to contain vasodilative activity as measured by the rapid and persistent induction of erythema in response to intradermal injection into rabbit skin. Tests for physical stability of the activities indicated that the vasodilators were proteinaceous and heat stable. The electrospray ionization mass spectroscopy of the S. vittatum protein detected a mass of about 15,000 Daltons.

Methods for the construction of S. vittatum salivary gland cDNA library and cloning of specific cDNA of Simulium vittatum salivary gland erythema protein (SVEP) were performed by the following steps:

SVEP was purified from salivary glands and sent to the Harvard Microchemistry Laboratory where it was subjected to limited digestion with trypsin. Two peptides, CT29 (SEQ ID NO: 3) and CT51 (SEQ ID NO: 4) were sequenced by an automated Edman degradation procedure.

Messenger RNA (mRNA) was isolated from SGE of S. vittatum. A commercially-available kit was used to prepare the cDNA library (ZAP EXPRESS™ cDNA synthesis kit, Strategene, La Jolla, Calif.).

A fragment of the SVEP cDNA was generated by PCR using degenerate primers that were designed based on knowledge of the partial amino acid sequence revealed in sequencing of the purified protein. A commercially available kit (TA Cloning® System, Invitrogen) was used to clone the PCR product. Sequencing of the cDNA and comparison of the translated amino acid sequence confirmed the validity of the clone. Further, the relative order of the two peptides and the intervening amino acids were determined (SEQ ID NO: 5). A digoxigenin-labeled (DIG) probe was generated for use in screening the cDNA library to recover the full-length clone.

Screening of the library produced a full-length clone that provided the remaining codes for all the amino acids, including a hydrophobic leader sequence that is cleaved from the mature, functional protein. Analysis of the remaining bases revealed that the mRNA for this protein has a relatively small number of non-translated base sequences at the N and C termini (SEQ ID NO: 1).

Calculation of the putative molecular weight of the mature protein which would be generated by the cDNA clone was 15,348.9 Daltons, which is 1.23 to 2.58 Daltons less than the weight of the HPLC purified protein determined by ESIMS.

Production of Recombinant SVEP Protein (rSVEP) via Baculovirus Expression System.

A commercially available baculovirus vector (pBacPAK8, Clontech Laboratories, Inc., Palo Alto, Calif.) and cDNA of SVEP were digested with PstI and Xhol restriction enzymes and religated to form a recombinant plasmid. Recombinant virus was produced by co-infection of Sf9 cells with the pBacPAK8/SVEP and baculovirus DNA digested with BSU361. Recombinant virus was purified by plaque assay and amplified. Production of SVEP DNA in the recombinant virus was confirmed by PCR amplification of cellular DNA isolated from infected cultures. Synthesis and secretion of protein of the appropriate molecular weight was demonstrated in SDS/PAGE of proteins present in the cellular cultures of recombinant-virus infected cells and absent from cellular supernatants of wild-type virus infected cells.

Quantitative Analysis of rSVEP

By examination of silver-stained, SDS protein gels, it was determined that rSVEP was >90% of the total protein in cell culture supernatants at 48 hr post infection.

Total protein concentration was determined using the Lowry method. Based on the observations of #1 above, rSVEP protein concentration was estimated as the difference between total protein concentration in cellular supernatants of BV/SVEP infected cells and wild-type infected cells.

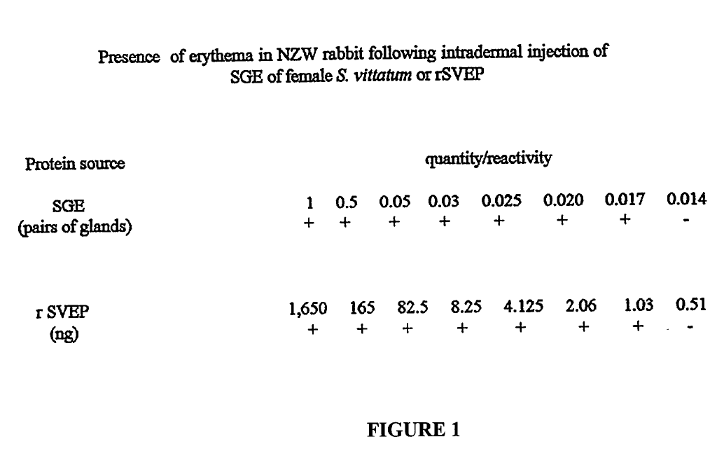

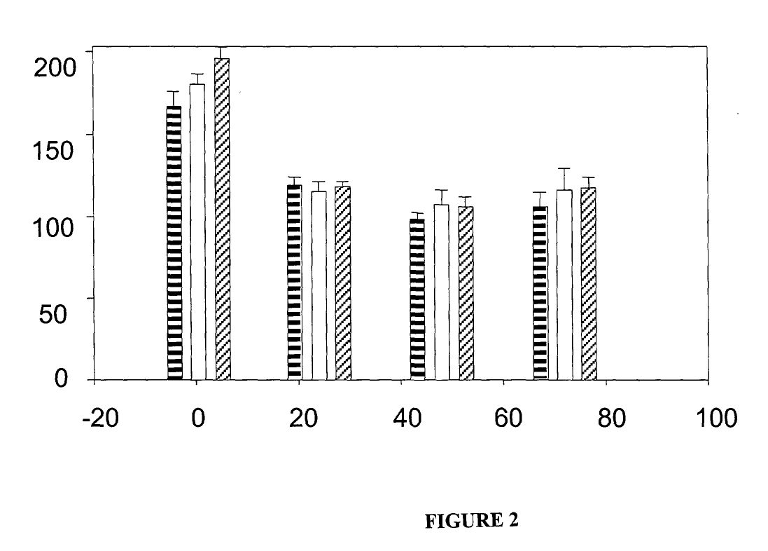

Using these quantitative measurements, the potency of rSVEP was estimated by bioassay in rabbit skin as described previously. The limit of detectable erythema following injection was approximately 1 ng, and was equivalent to the activity present in 0.017 pairs of S. vittatum salivary glands. For a protein of molecular weight 15,315 Daltons, this is equivalent to 65 femtomoles (FIG. 1).

Physical Properties of rSVEP

Native and recombinant SVEP have a compact tertiary structure that causes the protein to migrate at a faster rate, when subjected to gel sieving techniques, than would be predicted by molecular weight alone. Treatment with the disulfide reducing reagent, 2-mercaptoethanol, delays mobility and thus indicates that the two cysteines form a disulfide bond. Because these two amino acids are located at the two different ends of the sequence, substantial folding of the protein must occur to accommodate bond formation.

Amino acid composition of SVEP shows a relative high percentage of basic amino acids (see SEQ ID NO: 2; lysine, coded as K, and arginine, coded as R). Based on TSK gel sieving and protein staining patterns it is likely that the folded protein displays these basic moieties on its surface to produce a positively charged molecule.

Therapeutic uses of rSVEP

Test of rSVEP efficacy in facilitating and/or enhancing wound healing may be performed as follows.

Using NZW rabbits, sterile, surgical open and closed wounds will be created. rSVEP or control solution were injected intradermally or subcutaneously on a daily basis.

Differentiation of vasoactive effects from inflammation were determined using laser doppler imagery (LDPI) for reperfusion (described in more detail below) and histopathological evaluation for granulation tissue.

The rate of healing were determined using 3 measures: Planimetry to determine rate of open wound healing, histological evaluation to determine progression from inflammatory stage to repair stage, and tensiometry to determine strength of tissue repair.

### EXAMPLE 2

### Studies of SVEP Effects on Perfusion and Wound Healing

Increased perfusion in peripheral blood vessels in response to application of SVEP has been demonstrated in a number of animal species including NZW rabbits, diabetic Zucker rats, beagle dogs, and humans. The most extensive studies have been carried out with NZW rabbits where a dose-related increase in blood perfusion was measured that returned to control levels within 24 hours. See FIG. 2, which shows the dose-related perfusion response of NZW rabbits injected intradermally with SVEP. In spite of its relatively short duration of action, repeated daily injection of the protein for seven days resulted in a decrease in tissue necrosis in healing wounds when compared to PBS control-injected wounds (Wilcoxon Signed Ranks Test; n=6; p=0.059). A similar effect was demonstrated in diabetic Zucker rats even though concentration and total amount of SVEP treatment were lower (Wilcoxon Signed Ranks Test; n=16; p=0.096).

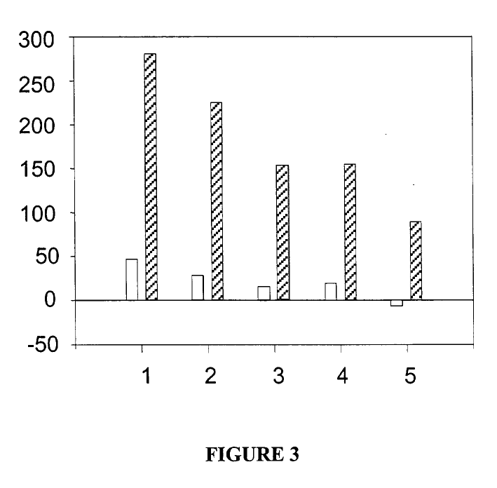

Later work testing blood flow response to intradermal injection of SVEP in beagle dog skin was found to be greater both in intensity and duration than the other species tested. See FIG. 3, which shows the perfusion response to SVEP in beagle dog skin. These results confirm that beagle skin also shows increased sensitivity of the perfusion response to SVEP.

### EXAMPLE 3

### Enhancement of Macrophage Activity by SVEP

The cell line RAW 264.7 was used as an in vitro model system to test the potential for SVEP to modulate macrophage activation responses. The RAW 264.7 cell line, derived from Mus musculus, was obtained from the American Type Culture Collection (ATCC TIB-71). This cell line was chosen because its macrophage-like properties have been well characterized and are known in the art.

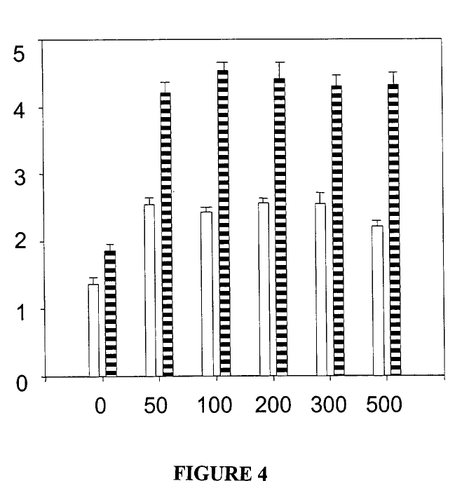

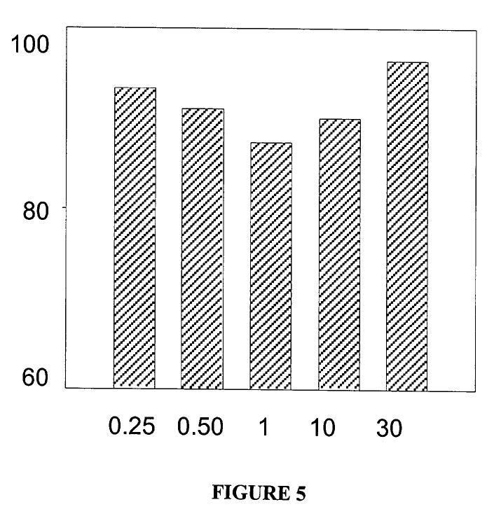

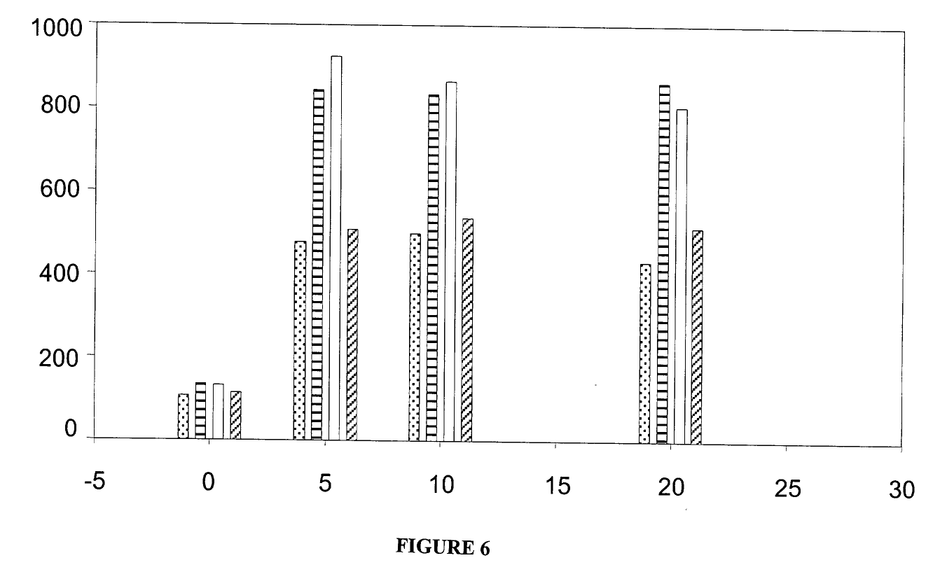

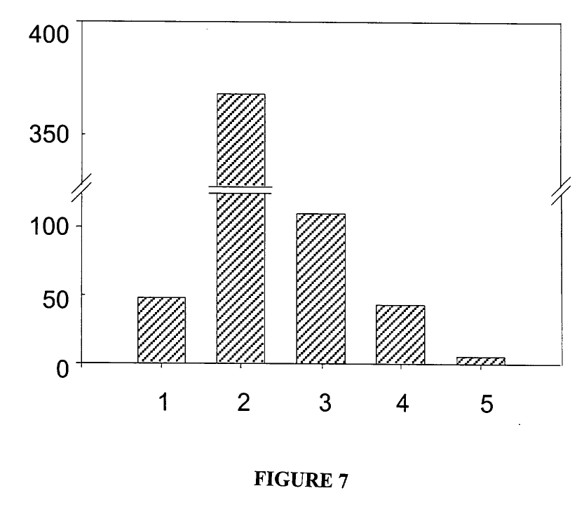

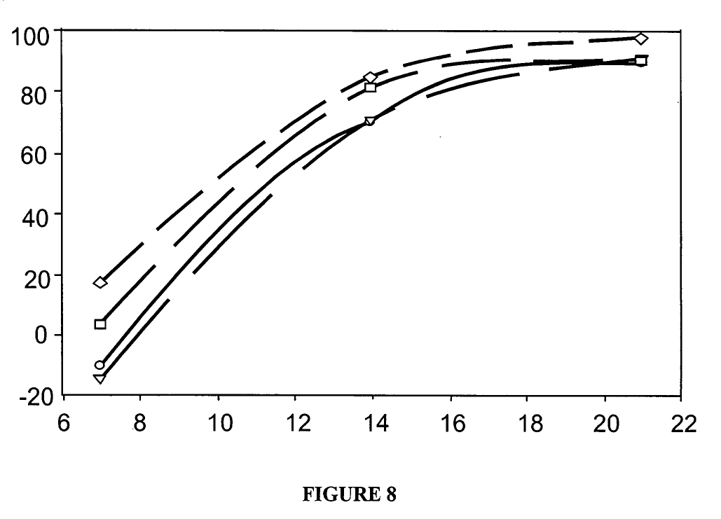

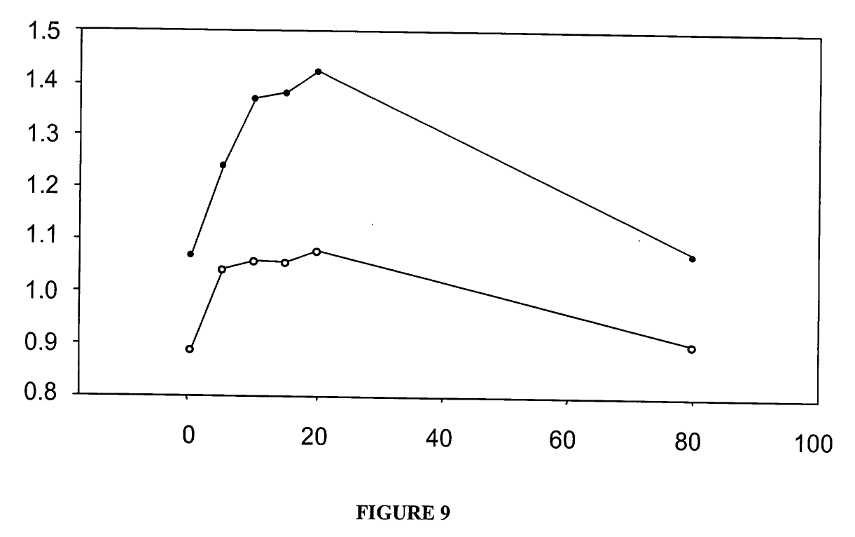

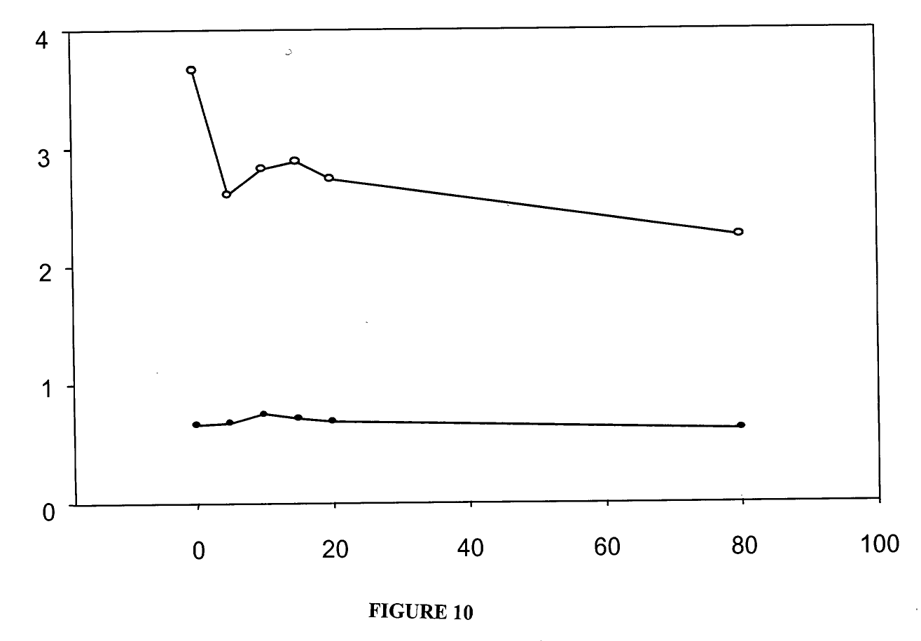

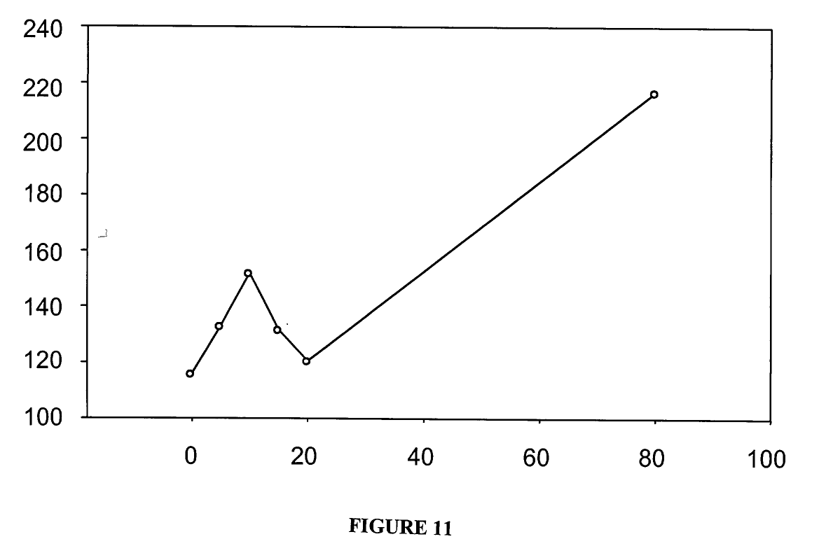

IFN-γ was obtained from R&D Systems, Minneapolis, Minn., which calibrates IFN-γ activity against the National Institutes of Health IFN-γ standard GG 23-901-530 as measured in anti-viral assays using HeLa cells infected with EMC virus (Meager (1987) Lymphokines and Interferons, a Practical Approach (Clemens et al., eds., IRL Press)). The response of cells to IFN-γ was determined by measuring the end product of NO released by activated cells (nitrite) in the cell culture medium at 24 and 48 hours post-treatment. For these experiments, one Unit of IFN-γ activity was defined as 0.25 nanograms. Cells were evaluated for activation response to doses of 0, 50, 100, 200, 300 or 500 Units/ml. All concentrations of IFN-γ tested caused macrophage activation that was maximal at 100 Units/ml. See FIG. 4, which shows the secretion of nitric oxide by macrophage-like cells (RAW 264.7) in response to increasing concentrations of IFN-γ.

An experiment was designed to test the effect of SVEP on the IFN-γ response of the model macrophage-like RAW 264.7 cells using the above-determined dose of IFN-γ (i.e., 100 Units/ml). Cells were treated for two hours with increasing concentrations of SVEP of 0, 0.25, 0.50 or 1 microgram per ml (16, 33, or 65 nanomolar) before the addition of 100 Units/ml of IFN-γ. This treatment resulted in an SVEP dose-related decrease in the secretion of NO by cells (see FIG. 5). Because, as discussed above, NO is a marker of macrophage response, this result shows that SVEP modulates the response of macrophage-like cells to IFN-γ. These experiments clearly demonstrate that, in vitro, SVEP directly impacts macrophage cellular response to IFN-γ. The interaction of SVEP and IFN-γ to modulate macrophage phenotypes in vitro provides an excellent model for addressing specific mechanism(s) of action related to its in vivo effect on tissue necrosis.

### EXAMPLE 4

### In vitro Analysis of Interaction Between SVEP and the Immune System

Optimal IFN-γ Concentration

RAW 264.7 cells growing in DMEM complete medium in 75 mm culture flasks were removed from the flasks by scraping and were collected by gentle centrifugation (1800 rpm×10 min). Cell viability was determined by the activation of fluorescein diacetate (FDA) (see Guilbault and Kramer (1964) Anal. Chem. 36: 409. Viable cells were plated at 5×10⁵ cells per well of a 24-well cell culture plate. After overnight culture to allow attachment, medium was removed and replaced with fresh DMEM complete medium containing IFN-γ in concentrations ranging from 0 to 100 Units/ml, with a minimum of four replicates each. Culture was continued for 48 hr, at which time the cell culture supernatant was removed and centrifuged to eliminate cellular debris. This clarified, cell-conditioned medium was then frozen for analysis of NO production by quantitative measurement of nitrite (see Ding et al.(1990) J. Immunol. 145(3): 940-944, which teaches analysis of NO production with assays utilizing the Griss reagent). The concentration of IFN-γ that was mid-point in the dose/response curve was chosen as the standard dose for macrophage activation in further studies.

Cell Viability and Proliferation

Another procedure was performed to measure the relative number of surviving cells following the 48-hour incubation period. This “MTT” (methylthiazoletetrazolium) assay involves exposing the cells to a chemical dye (3-[4,5-dimethylthiazol-2-yl]-2,5-diphenyltetrazolium bromide) which is taken up by viable cells and metabolized to a chemical that absorbs at a different wavelength (see Mosmann (1983) J. Immunol. Methods 65:55-63). Thus, the absorbance of the dye metabolite is a function of the number of viable cells and thus the viable cell number can be estimated. The MTT assay showed that, in comparison to the no-SVEP control, SVEP treatment at concentrations between 5 and 20 nanomolar provided a cell proliferative response. (see FIG. 9) However, at the highest concentration of SVEP used, the cell proliferative response decreased so that cell numbers were comparable to control levels. This result suggests that SVEP may improve wound healing by the additional mechanism of stimulating cell proliferation. Absorbance values of cells are shown as a function of SVEP concentration (nanomolar).

Optimum SVEP Concentration

To determine the optimum concentration of SVEP that inhibited in vitro macrophage activation, cells were treated with the standard dose of IFN-γ determined earlier and were also treated with concentrations of SVEP between 5 and 80 nanomolar. This analysis showed that NO production was inhibited by SVEP, demonstrating the deactivation of an inflammatory macrophage phenotype. (see FIG. 10).

Production of IGF-1

The presence of IGF-1 was confirmed by further ELISA analysis of the clarified, cell-conditioned medium from this experiment. Results shows that the deactivation by SVEP was correlated to an increase in the production of IGF-1 that is associated with a healing phenotype. (see FIG. 11).

### EXAMPLE 5

### SVEP Promotes Wound Healing

To test the effect of SVEP on blood flow in dogs, two kinds of wounds were created: closed wounds (i.e., sutured wounds) and open wounds (i.e., non-sutured).

Matched pairs of wounds were treated with SVEP in a buffer solution or a control buffer solution without SVEP, and healing was assessed by objective measurements, for example, by assessment with Laser Doppler Perfusion Imaging (LDPI). LDPI is capable of quantitative measurement of blood flow by detecting perfusion of blood in an imaged area.

Examination of sutured wounds treated with SVEP showed that SVEP treatment produces stronger repair of closed wounds. The median breaking strength on day 5 of matched wounds increased by 48 percent when treated with SVEP. Breaking strength was assessed by removing a segment of skin from the area of a sutured wound including a portion of the sutured wound. The area from which the skin was taken was sutured and allowed to heal. The removed skin was placed in a tensiometer, and tension was applied to determine the breaking strength of the wound.

SVEP treatment also produced faster repair of open wounds, increasing healing by an average of 14 percent by day 21 when SVEP was intradermally injected on day 0.

### EXAMPLE 6

### Increase in Skin Perfusion of Dogs in Response to SVEP

Recombinant SVEP was produced in a baculovirus protein expression system (BD Biosciences Clontech, Palo Alto, Calif.) and HPLC-purified. Control solutions and SVEP solutions were prepared in Phosphate Buffered Saline (PBS) and filtered with a 0.22 micron filter to sterilize the solutions.

Approximately twenty-four hours prior to testing, the dorsolateral aspect of the trunks of four beagle dogs was clipped. One dog was tested at a time, and food was withheld from each dog four hours prior to each sedation. Under appropriate sedation, four sites on each side of the dog were marked for testing with a fine-tipped marker and time 0 readings of perfusion obtained. A range of SVEP dosages (0, 5 10, and 20 μg in 50 microliters PBS) was injected into the prepared area on each side of the dog but the order of lowest to highest dose was reversed in the cranial to caudal direction in order to account for any endogenous cranial to caudal perfusion differences. LDPI readings were taken at times 0.5, 24, 48, and 72 hours post-injection to follow duration of response. Only light sedation was required to obtain these subsequent readings. Percent increase in blood flow was calculated from measurement of flow at the same site prior to injection (time 0).

The results, which are presented in FIG. 6, show that blood flow was increased at all concentrations of SVEP tested. The blood flow reached maximum levels by 24 hours and declined to 0.5 hour levels at 72 hours. Blood flow was significantly higher at both 0.5 and 72 hours than at time 0. An earlier trial in a beagle dog showed a similar pattern, and blood flow decreased to time 0 levels at 96 hours.

### EXAMPLE 7

### Breaking Strength of Closed Wounds

Surgical Procedures

Six purpose-bred beagle dogs were used in this study and preoperative procedures were generally the same as those described above. Dogs were preanesthetized prior to surgery with atropine sulfate (0.05 mg/kg body weight) and butorphanol tartrate (0.2 mg/kg body weight) followed by anesthetization with medetomidine (0.2 mg/kg body weight). After placing the dog in sternal recumbency, the dorsolateral trunk area was clipped and prepared for aseptic surgery from the level of the scapulae to the tuber coxae. Using aseptic technique, 3 sets of paired wounds were made on each side of the dorsal midline. At the level of the 13th rib, bilateral full-thickness square defects 2 cm on a side were created. Each wound was 8 cm ventrolateral to the dorsal midline. These wounds were used for histopathologic and electron microscopic analyses. Eight cm cranial to the open wounds, bilateral 3 cm long full-thickness skin incisions were made parallel to the dorsal midline and 8 cm ventrolateral to it. These wounds were sutured with 4 equidistantly placed simple interrupted sutures of 3-0 polypropylene. These wounds were used for LDPI and breaking strength determination. Eight cm caudal to the open wounds, another set of bilateral square open wounds 2 cm on a side were created 8 cm ventrolateral to the dorsal midline. These wounds were used for LDPI and planimetric evaluation. On three dogs the wounds on the left side were treated with SVEP and the wounds on the right were treated identically with PBS as controls. On the other three dogs the treatment and control sides were reversed.

Initially, three dogs were treated with intradermal injections and three dogs were treated by subcutaneous injection. This procedure was used on day 0 when dogs were sedated. However, on the subsequent treatment days (days 3, 6, 9, 12) when the dogs were not sedated, injection of the wound appeared to cause some pain, so subsequent injections were only performed subcutaneously. Healing of the open wound on day 21 was improved significantly in those that received the day 0 intradermal injection and perhaps would have shown even better results had that method of delivery been maintained. Those dogs that received all subcutaneous treatments did not show significant improvement in healing of open wounds, measured at day 21, but did have stronger repair of closed wounds.

After the wounds were made and the incisions sutured, an LDPI scan was performed on the 2 cranially located sutured wounds and the 2 caudally located sutured wounds. These served as base line data. The treated open wounds were injected in the subcutaneous tissues with the selected SVEP dose in 50 microliters of PBS (i.e., 5 micrograms of SVEP in 50 microliters of PBS), injecting four equal fractions of the total dose along each border of the square wounds. The sutured wound was injected in the subcutaneous tissue with a like dose. Control wounds were injected in like manner with 50 microliters of PBS. After 30 minutes the LDPI scans were repeated on the cranial (sutured) and caudal (open) wounds. The caudal open wounds were traced on clear acetate.

Dogs were bandaged with a body bandage. Bandages were changed daily for the first 10 days then every other day until 21 days. Injection of SVEP and PBS had been planned as described above on days 2, 4, 6, 8 and 10, but this timetable was changed based on a dose/response experiment that showed the response was maximally elevated for 3 days. Thus, injection of SVEP and PBS was performed on days 3, 6, 9, 12. The dogs were anesthetized on days 0, 5, 7, 14 and 21. An 8 mm diameter biopsy was taken from the 2 middle open wounds at day 5. The biopsy was cut in half. One half was evaluated using light microscopy and the other half was evaluated with electron microscopy. The resulting defect after removal of the biopsy for histopathology and electron microscopy is allowed to heal as an open wound.

On day 5 while the dogs were under general anesthesia, the 2 sutured wounds (one treated and one control) were harvested for breaking strength analysis. Preanesthesia was with acetylpromazine (0.1 mg/kg body weight, injected intramuscularly) and atropine sulfate (0.05 mg/kg body weight, injected subcutaneously). Anesthesia was induced with thiopental (12 mg/kg body weight, administered intravenously). After intubation, anesthesia was maintained with 2% isoflurane in 30 mls: 02/kg body weight/min. On days 0, 5 and 14, open wounds were evaluated with LDPI. Sutured wounds were evaluated with LDPI on days 0 and 5. On days 0, 7, 14 and 21 planimetric measurements were made of the wounds. For the first 10 days, the dogs were administered an antibiotic-cefadroxil (22 milligrams per kilogram body weight every 12 hours, by mouth). Any postoperative discomfort was treated with butorphanol tartrate (0.2 mg/kg body weight, injected subcutaneously.

Evaluation Procedures and Data Analysis

LDPI is used to evaluate tissue perfusion. LDPI is a noninvasive technique that uses a low power He—Ne laser beam to sequentially scan a tissue surface. The laser beam is directed to the tissues and reflected back from both stationary and moving tissue (red blood cells). A scanning device then computes the percentage of reflected beam coming from moving tissue (red blood cells). This gives an indication of tissue perfusion for the scan site. The more red blood cells that are present and moving, the higher the perfusion percentage. The LDPI technique makes it possible to study the spatial variations in tissue perfusion at discrete times.

With the dogs under the influence of dormitor and tobugesic, laser Doppler perfusion imagery (LDPI) was used to assess wound perfusion immediately before and 30 minutes after treatment with SVEP or PBS on Day 0 and four additional times on subsequent days (days 5, 7, and 14). A scan area that encompassed the entire open wound plus an edge of intact skin on each side of the newly created wound was selected as the standard scan area. All subsequent LDPI scans covered the same standard area, centered on the healing wound, even though that wound was expected to decrease in size due to wound contraction. A similar sized area of sutured wounds was scanned with the wound in the center of the area.

Change in perfusion was determined by difference in LDPI readings between Time 0 and subsequent time points measured. Analysis identified the lowest dosage that induced the most sustained perfusion response (5 micrograms of SVEP in 50 microliters of PBS) and this concentration was used in the wound-healing tests.

Breaking strength was determined by removing a 2×5 cm rectangular segment of skin from the area of the sutured wounds with a 2 cm length of the sutured wound bisecting the segment across its short dimension. The segment of skin was placed in a tensiometer and tension was applied to determine the breaking strength of the wound. The resulting defect after removal of the skin segment was sutured and allowed to heal.

Wound healing was assessed by planimetric evaluation. Following sedation of the dogs with domitor and torbugesic, outlines of the wound edges and the advancing edges of epithelium of the caudal-most wounds were traced on clear acetate at days 0, 7, 14, and 21. The tracings were used with a computer digitizing program to determine total wound area, area of epithelium on a wound and area of unepithelialized granulation tissue. The area of unhealed wound and the area of epithelium were used to calculate the following values: Contraction, which is the percent of wound contraction at each time of measurement compared to the original wound area; Epithelialization, which is the percent of each wound that is epithelialized on each day of measurement; and Total Wound Healing, which represents combined contraction and epithelialization at each time of measurement compared to the original wound size.

Results

Results of breaking strength tests are shown in FIG. 7. The breaking strength of closed wounds was measured at day 5. SVEP treatment improved the breaking strength of closed wounds (Wilcoxon Signed Rank Test, n=5, p=0.043). The median increase in breaking strength due to SVEP treatment was 48%, thus demonstrating that SVEP treatment improves the breaking strength of closed wounds.

Data for Dog 6 was omitted as an outlier, although the breaking strength of the SVEP treated wound was dramatically stronger than that of the PBS-treated control wound because the possibility could not be excluded that the apparent difference was affected by the size of the closed wounds removed for measurement from Dog 6, which were accidentally cut shorter than the others.

Results for total healing of open wounds are shown in FIG. 8. Results show that treatment with SVEP with the intradermal/subcutaneous regimen resulted in a significant overall improvement in wound healing (paired T test, SVEP intradermal/subcutaneous data versus PBS intradermal/subcutaneous data), even though the intradermal method of application was applied only for the first treatment at day 0. Treatment by the intradermal/subcutaneous regimen improved wound healing over the subcutaneous regimen for both PBS (paired t test, p=0.045) and SVEP (paired t test, p=0.022).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed is:

1. A method for treating a wound in a mammal, said method comprising administering a therapeutically effective amount of a protein comprising an amino acid sequence which is at least 90% identical to the mature form of the amino acid sequence of SEQ ID NO:2, wherein said protein promotes wound healing.

2. The method of claim 1, wherein said protein comprises a first amino acid sequence which is at least 95% identical to the mature form of the amino acid sequence of SEQ ID NO:2.

3. The method of claim 2, wherein said protein is produced by recombinant methods.

4. The method of claim 1, wherein said protein comprises an amino acid sequence which is the mature form of the amino acid of SEQ ID NO:2.

5. A method for treating a wound in a mammal, said method comprising administering a therapeutically effective amount of a protein comprising an amino acid sequence which comprises at least 50 contiguous amino acids of the amino acid sequence of SEQ ID NO:2, wherein said protein promotes wound healing.

6. The method of claim 5, wherein said protein comprises an amino acid sequence which comprises at least 100 contiguous amino acids of the amino acid sequence of SEQ ID NO:2.

7. The method of claim 1, wherein said mammal is a human patient.

8. The method of claim 7, wherein said human patient is diabetic and said wound is an ulcer.

9. The method of claim 7, wherein said human patient has undergone surgery and said wound is a surgical wound.

10. The method of claim 7, wherein said wound is a result of blunt force injury.

11. The method of claim 7, wherein said wound is a result of heart failure.

12. The method of claim 1, wherein said mammal is a dog.

13. The method of claim 1, wherein said protein is administered by intradermal injection.

14. The method of claim 1, wherein said protein is administered topically.

15. The method of claim 1, wherein said protein is administered in combination with at least one other compound intended to promote wound healing.

16. The method of claim 15, wherein at least one of said other compound is an antibiotic and wherein said antibiotic is administered orally.

17. The method of claim 1, wherein said wound is an ocular wound.

18. The method of claim 1, wherein said protein also enhances the healing macrophage response.

19. A method for decreasing inflammation, said method comprising administering a therapeutically effective amount of a protein comprising an amino acid sequence set forth in SEQ ID NO:2, wherein said protein decreases inflammation.
